(19) United States
(12) Patent Application Publication
(10) Pub. No.: US 2019/0343536 A1
(43) Pub. Date: Nov. 14, 2019

(54) PRESSURISABLE WRIST BAND FOR ACHIEVING PATENT HEMOSTASIS OF AN ARTERIOTOMY

(71) Applicant: Biolife, L.L.C., Sarasota, FL (US)

(72) Inventors: Talmadge Kelly Keene, Ruskin, FL (US); Michael William Hudson, Bradenton, FL (US)

(21) Appl. No.: 16/388,674

(22) Filed: Apr. 18, 2019

Related U.S. Application Data
(60) Provisional application No. 62/659,336, filed on Apr. 18, 2018.

(51) Int. Cl. A61B 17/135

(52) U.S. Cl. CPC A61B 17/1355; A61B 2017/00022

(57) ABSTRACT

An air pressure-controlled inflatable compression device designed to be worn around the wrist to supply sufficient compression to control bleeding from an arteriotomy in the radial artery, while allowing blood to flow through the radial artery from the heart to the hand is provided. The device contains a bladder with a controlled inflation mechanism and a pressure sensor or indicator. The desired inflation pressure will be determined based on the blood pressure of the patient. The bladder pressure may be determined by a clinician based on the patient's vital statistics or the device may index the pressure using a pump and valve based on an electronic blood pressure sensor and a microprocessor controller. The length of time that the band compresses the artery will be determined by a clinician or microprocessor controlled.

### RELATED APPLICATIONS

The present application claims priority to U.S. Provisional Application No. 62/659,336, filed on Apr. 18, 2018, the entire disclosure of which is incorporated by reference as if set forth verbatim herein.

### FIELD OF THE INVENTION

The present invention relates in general to hemostasis, and in particular to hemostasis of the radial artery while maintaining blood flow distal to the arteriotomy in the artery.

Specifically, it relates to a device for performing hemostasis using controlled compression as related to air pressure within a bladder-containing cuff while maintaining patency (so called, “patent hemostasis”).

### BACKGROUND OF THE INVENTION

Arterial catheterization procedure sites are moving from the femoral artery to the radial artery. Procedures accessing the radial artery are referred to as transradial access (TRA). In some countries in Europe, catheterizations are over 80% via TRA, and in the United States, this number has grown to over 40% in recent years and is growing rapidly. The advantages of radial, over femoral, access include less frequent and less severe post procedural complications. However, with the absence of femoral vascular closure devices, radial hemostasis takes longer, resulting in radial artery occlusions (RAOs). RAOs are the most common and significant complications in radial procedures. RAO incidence as high as 10% of all TRAs has been reported in some studies. There are two main arteries that supply blood to the hand of the body. The radial artery is near the thumb and the ulnar artery is on the opposite side of the wrist. Losing blood flow to the hand from either of these arteries is problematic.

RAOs are more prevalent in persons with smaller arteries and are more prevalent with hemostasis procedures that hold the artery completely closed (occluded) during the hemostasis procedure for an extended amount of time. There are many studies in this field related to reducing hemostasis time to reduce incidents of RAOs. In addition sheaths have been engineered with hydrophobic coatings to reduce friction in an attempt to limit damaging the internal walls of the vessel.

One method of holding compression post procedure is for the clinician to apply manual pressure. Because the bleeding is from an artery and the patients have been given blood thinners to prevent clot formation, compression devices have also been developed to hold compression over the artery.

During sheath removal with typical bands available in today's market the artery is compressed to the point of occlusion and then the sheath is removed. This initial compression ensures that the vessel walls are tightly compressed onto the sheath as it is being removed but traumatizes the vessel walls. In addition, there is no feedback from known bands that provide the amount of compression that the band is applying to the patient.

### SUMMARY OF THE INVENTION

Briefly, the invention is a method and a device for titrating pressure over an artery with post transradial procedure with sufficient compression force to stop the bleeding from the arteriotomy, but with not so much force so as to close off the vessel. By not compressing the artery with occlusive compression, the prevalence of RAOs will be dropped to a minimum. In addition, not overly compressing the artery during the sheath removal (post procedure) will minimize trauma to the inner vessel walls and minimize damage to the ulna nerve and ulnar artery. Allowing blood to flow through the artery during the healing of the arteriotomy will hasten healing of the arteriotomy. Using this device will provide safer, and better, care for the patient.

### BRIEF DESCRIPTION OF THE DRAWINGS

FIG. 1 is a schematic representation of a person's arteriotomy location on the arm;

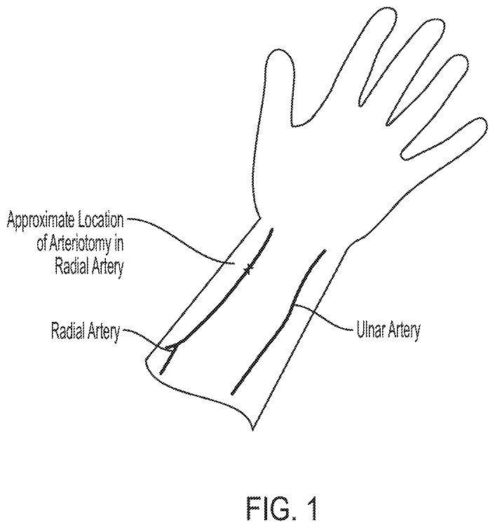

FIG. 2 is a schematic representation of various embodiments of the present invention, including various bladder configurations for use in the present invention;

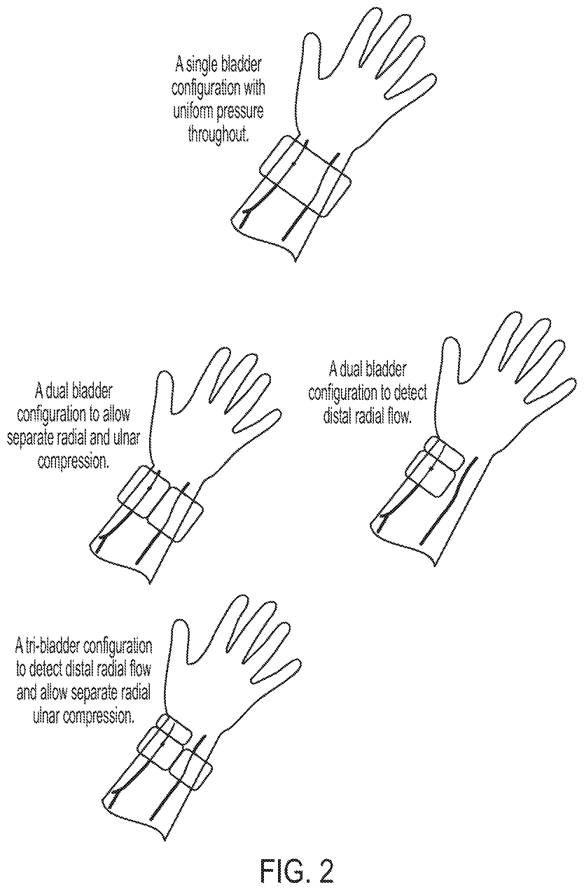

FIG. 3 is a schematic representation of another embodiment of the present invention; and

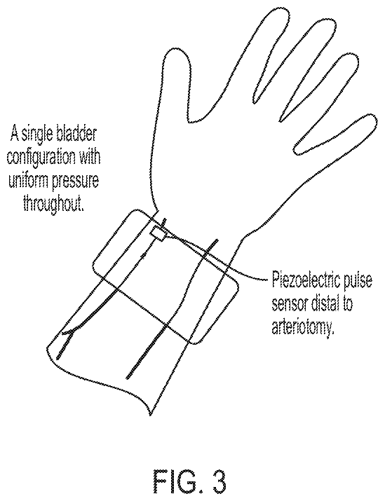

FIG. 4 is a schematic representation of additional embodiments of the present invention.

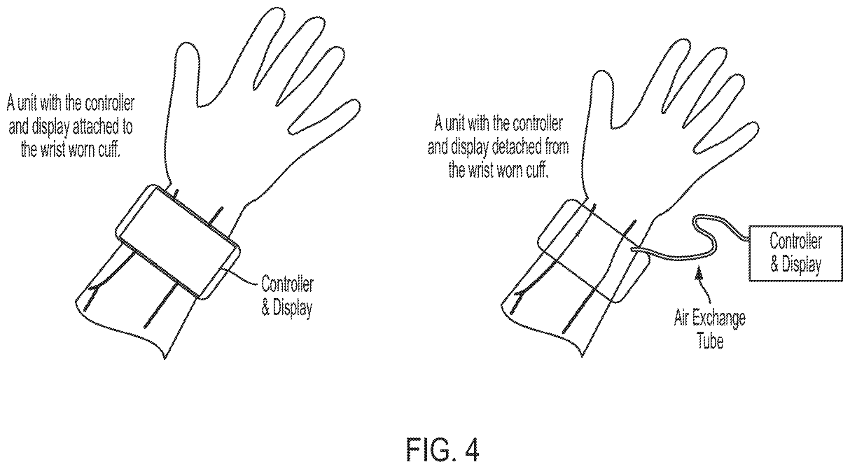

### DETAILED DESCRIPTION OF PREFERRED EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. The following embodiments and aspects thereof are described and illustrated in conjunction with systems, tools and methods which are meant to be exemplary and illustrative and not limiting in scope. In various embodiments one or more of the above-described problems have been reduced or eliminated while other embodiments are directed to other improvements.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

The present invention now will be described more fully hereinafter with reference to the accompanying drawings, in which some, but not all embodiments of the invention are shown. The present invention may be embodied in many different forms and should not be construed as limited to the embodiments set forth herein; rather, these embodiments are provided so that this disclosure will satisfy applicable legal requirements and demonstrate exemplary embodiments of the invention. Repeat use of reference characters in the present specification and drawings is intended to represent same or analogous features or elements of the invention. A full and enabling disclosure of the present invention, including the best mode thereof directed to one of ordinary skill in the art, is set forth in the specification, which makes reference to the appended drawings.

The present inventive device consists of a cuff-like device typically used for measuring blood pressure. Within the cuff is an air bladder. Prior to removal of the sheath, the cuff is secured around the patient's wrist. Then, just prior to removal of the sheath, the air bladder is inflated to an operational pressure to provide patent hemostasis or stoppage of the blood flow (hemostasis) from the arteriotomy site (hole in artery created during TRA) while allowing blood to flow through the artery (patency).

The illustrations below are examples of arterial compression. With “Over Compression”, the artery will be closed and will not allow blood to pass through the artery, bleeding from the arteriotomy will be controlled, and ROA formation is possible. With “Under Compression”, the artery will allow blood to pass through the artery, but the artery will leak blood from the arteriotomy due to lack of compression and there will be no ROA formation. This blood may exit the body through the dermatotomy or be captured between the vessel and the skin, resulting in a hematoma. “Good Compression” will result in control of bleeding from the arteriotomy while allowing blood to pass through the artery (patent hemostasis). Operation pressure of the inventive band will result in “Good Compression”. The effects and complications, as well as illustrations, of Over, Under, and Good Compression are set forth in Table 1.

If a manual system is employed, then operational pressure could be determined based on MAP (mean arterial pressure), or a simpler system may be to simply use SBP (systolic blood pressure) minus a predetermined mmHg value or DBP (diastolic blood pressure) plus a predetermined mmHg value.

Operational pressure is expected to be Mean Arterial Pressure (MAP):

(MAP)=DBP+((SBP−DBP)/3)

Using the patient's vital signs (SBP, DBP, MAP) to set a desired pressure is a novel approach to transradial hemostasis. Some clinicians may prefer to do a series of stepdown deflations protocol versus time. There is potential that at some point during the procedure, the compression may be reduced to a point below DBP.

An improvement to the inventive system would be to employ a microprocessor-controlled system with an electric pump, valve, and pressure sensor. One of ordinary skill in the art would be able to add such a control system to the present invention.

The device could then automatically detect the patient's blood pressure and set the inflation operational pressure accordingly. The microprocessor-controlled system has many benefits other than simply ease of use. For example, during the procedure, the band could reinflate to check the patient's blood pressure. The band could have a timer and alarms. The band could have a series of steps programmed into the device for deflation. The device could have wireless communication capabilities. There may be separate bladders to control the ulnar artery flow. There may also be a need to confirm that the patient has blood flow distal to the arteriotomy. The microprocessor-controlled device may have manual overrides as deemed necessary by the attending clinician.

There also may be a need to create a device with the controller attached, and a device with a separate controller. The separate controller design may lead to a potentially reusable device allowing for a more expensive and complete system, with more electronics in the controller. A manual valve, may be employed, on the air line from the cuff to the controller to allow the controller to be removed and maintain operational pressure of the inflated bladder. All of such improvements could be made to the present invention by those of ordinary skill in the art.

The present inventive device may also be used in conjunction with hemostasis-assisting technology, such as hemostasis pads.

This technology could be transformed to perform patent hemostasis on any near-surface arteries of the body, given the right device design. If the bladder or bulb is too rigid or puts back pressure on the system, then the back pressure should be taken into account for inflation purposes to acquire the proper compression.

The bladder used in the system should avoid adding significant back pressure as it is being inflated for normal usage. Otherwise, the volume of air versus bladder back pressure would need to be determined and included in the calculation based on inflation volume.

The pump designed for the system should not excessively leak air. Otherwise, a check valve could be employed.

A device could be designed whereby the pump continually, one time, or intermittently runs or engages and the valve simply operates as a pressure relief system. For example, the valve would be set to relieve pressure at 100 mm of Hg and the pump designed to inflate the bladder to above 100 mmHg based on pressure capabilities, volume of air delivered and time at which the pump is running. The valve could be a preset design or an adjustable valve. The pump may be manual or electric. The system may or may not have a pressure display.

The manually-operated device of the present invention may operate as follows. First, the patient's blood pressure is taken prior to the procedure. Vascular procedure is then completed. Wound site is prepared for cuff application. Cuff is placed around the patient's wrist. The clinician analyzes the patient's blood pressure (taken prior to the procedure) and inflates the cuff to mean MAP blood pressure. The inflation pressure may also be based on SBP or DBP. The sheath is pulled. The clinician notes the time. After a predetermined amount of time, the cuff is deflated and removed. The site is observed for bleeding or hematoma formation.

At this point, the clinician may confirm the artery is patent.

Some clinicians may choose to over inflate initially and set a deflation protocol over times. Some clinicians may choose not to fully deflate and, instead, perform a stepwise deflation protocol. The inflation/deflation protocol could be accomplished with a manual pump and valve or an electronic pump and valve.

The microprocessor-integrated device of the present invention may operate as follows. First, the patient's blood pressure is taken prior to the procedure. Vascular procedure is then completed. Wound site is prepared for cuff application. Cuff is placed around the patient's wrist. The clinician powers on the band and, thereafter, presses the start button.

The microprocessor engages the pump with the valve closed and automatically inflates above typical SBP based on feedback from the pressure sensor. Typically, the initial inflation will be in the 150-200 mmHg range. The band could also be designed to inflate and stop to determine approximate DBP to set operational pressure. Or, the band could be designed to determine both SPB and DBP prior to setting the operation pressure.

The microprocessor turns off the pump and opens the valve. The sensor monitors the deflation until a fluctuation in air pressure is identified. This fluctuation is due to the SBP of the patient.

The microprocessor calculates a band set point pressure at approximately 60-90% of the SBP of the patient. This is the operation set point.

With the pump off, the valve deflates the band to the operation set point. Periodically (for example, every 5 mins) the device checks the pressure and inflates or deflates the bladder as needed to maintain the operation set point.

At a predetermined time (for example, 60 minutes) the microprocessor sounds an alarm indicating that the cuff is ready to be evaluated for removal. The device displays the pressure readings and a digital timer for the clinician to observe. The clinician deflates the band and checks the patient for bleeding or hematoma formation.

The clinician or device may confirm the artery is patent.

The cuff may need mechanical overrides to allow the clinician to set the pressure accordingly with their protocol. The band may be designed to assess the patient blood pressure periodically and reset operational pressure. In addition, there may be some clinical benefits to pulsating the artery during the procedure.

Various features include: 1) Operation pressure may be a single set point or step down during the procedure; 2) There may be other alarms integrated in the system to identify abnormalities in the patient's blood pressure readings; 3) Other devices may be integrated with the cuff; 4) A manual valve may be employed between a stand along controller and the cuff to allow the controlled to be disconnected from the cuff while the cuff remains inflated to operational pressure; 5) Wireless technology may be incorporated with the cuff; 6) There may be some need to hold the band at near or above SBP for a short time prior to the device beginning the deflation process.

### EXAMPLE 1

Using a hand pump and a disassembled wrist blood pressure cuff, it was found that at a blood pressure in two persons of 120/70 mmHg and 130/80 mmHg, respectively, that at 100 mmHg inflation pressure using ultrasound strong blood flow was determined. From the systolic blood pressure to 100 mmHg the sound signal could be heard to be increasing in strength, but below 100 mmHg there was no audible difference in flow based on the ultrasound signal. The 100 mmHg amount of compression should be sufficient for patent hemostasis.

What is claimed is:

1. An air pressure-controlled compression device for controlling bleeding, comprising:
a securement cuff configured to be wrapped around a body part of a patient that is bleeding as a result of post arterial access procedures at an access site;
an air bladder attached to the cuff positioned over the access site;
an air compressing pump in pneumatic communication with the bladder;
an optional check valve;
a pressure sensor attached to the cuff in pneumatic communication with the pump;
a pressure relief valve in communication with the bladder;
a control unit in electrical communication with the pump, the sensor, and the valve;
a power source connected to the control unit;
said control unit including a timer;
wherein, when the cuff is wrapped around the body part, the control unit is configured for activating the air pump to pressurize the air bladder above systolic blood pressure to fully occlude the radial artery of a patient,
wherein, the control unit is configured to deactivate the pump once the bladder is fully pressurized above systolic blood pressure and activate the valve to release pressure;
wherein, the pressure sensor is configured to detect the systolic blood pressure as the artery begins to pulse as compression is reduced during bladder deflation,
wherein, the control unit is configured to calculate an operational inflation pressure at a point at below the measured systolic blood pressure to hold compression that will control bleeding external to the artery and skin while allowing blood to flow through the artery to the tissue distal to the arterial access site;
wherein, the control unit maintains bladder pressure at operational inflation pressure by activating the pump or valve at operational inflation pressure as regulated by the pressure sensor and microprocessor timing or operational pressure range settings.

2. The device of claim 1 further comprising a first additional bladder.

3. The device of claim 2 further comprising a second additional bladder.

4. The device of claim 1 comprising a piezoelectric device located over the radial artery to detect radial artery pulsation distal to the arteriotomy.

5. The device of claim 1 comprising manual overrides to control inflation pressure or timing.

6. The device of claim 1 wherein the securement cuff and bladder are transparent in order to allow viewing of the area of the skin around the arteriotomy and dermatotomy to detect bleeding or hematoma formation.

7. The device in claim 1 comprising moisture sensing capabilities to detect active bleeding.

8. The device in claim 1 comprising hematoma detecting capabilities.

9. An air pressure-controlled compression device for controlling bleeding, comprising:
a securement cuff configured to be wrapped around a body part of a patient to control bleeding post arterial access procedures;
an air bladder attached to the cuff;
an air pump in pneumatic communication with the bladder;
an optional check valve;
a pressure gauge attached to the cuff in pneumatic communication with the pump;
a pressure relief valve in communication with the bladder.
